(19) United States
(12) Patent Application Publication
(10) Pub. No.: US 2009/0028816 A1
(43) Pub. Date: Jan. 29, 2009

(54) Treatment of depression, psychosis, and anxiety

(76) Inventors: Lance Sultzbaugh, Alameda, CA (US); Kirk W. Johnson, Moraga, CA (US)

Correspondence Address: ROBINS & PASTERNAK LLP, 1731 EMBARCADERO ROAD, SUITE 230, PALO ALTO, CA, 94303, US

(21) Appl. No.: 12/220,203

(22) Filed: Jul. 22, 2008

Related U.S. Application Data
(60) Provisional application No. 60/962,360, filed on Jul. 27, 2007.

(51) Int. Cl. A61K 31/437; A61K 31/5513; A61K 38/20; A61P 25/00; A61P 25/24; A61P 25/18

(52) U.S. Cl. 424/85.2; 514/300; 514/221

(57) ABSTRACT

The use of ibudilast (3-isobutyryl-2-isopropylpyrazolo[1,5-a]pyridine) for treating affective disorders, such as depression, psychosis, or anxiety, is described.

### CROSS-REFERENCE TO RELATED APPLICATION

This application claims the benefit under 35 U.S.C. §119(e)(1) of provisional application 60/962,360, filed Jul. 27, 2007, which application is hereby incorporated by reference in its entirety.

### FIELD OF THE INVENTION

The present invention relates generally to methods of treating affective disorders, including symptoms of depression, psychosis, and anxiety. In particular, the present invention pertains to methods of treating or preventing depression, psychosis, or anxiety by administration of a glial attenuator, phosphodiesterase inhibitor, and leukotriene D4 synthesis inhibitor, such as ibudilast (3-isobutyryl-2-isopropylpyrazolo[1,5-a]pyridine; also termed AV411 herein).

### BACKGROUND OF THE INVENTION

Affective disorders, the most common psychiatric disorders in adults, are characterized by changes in mood as the primary clinical manifestation. Such disorders include depression, bipolar disorder, post-partum depression, dysthymia, seasonal affective disorder, schizoaffective disorder, general anxiety disorder, panic disorder, and posttraumatic stress disorder. In cases where disturbances in mood (depression, anxiety, elation, and excitement) are severe, patients may additionally experience psychotic symptoms.

Major depression is manifested by a combination of symptoms that interfere with the ability to work, study, sleep, eat, and enjoy once pleasurable activities. Symptoms of depression may include persistent sad, anxious, or “empty” mood, feelings of hopelessness, pessimism, guilt, worthlessness, or helplessness, loss of interest or pleasure in hobbies and activities that were once enjoyed, including sex, decreased energy, fatigue, and a sense of being “slowed down,” restlessness, irritability, difficulty concentrating, remembering, or making decisions, sleep disturbances, such as insomnia, early-morning awakening, or oversleeping, loss of appetite and/or weight loss or overeating and weight gain, thoughts of death or suicide and/or suicide attempts, and persistent physical symptoms that do not respond to treatment, such as headaches, digestive disorders, and chronic pain. A disabling episode of depression may occur only once but more commonly occurs several times in a lifetime. A less severe type of depression, dysthymia, involves long-term, chronic symptoms that do not disable, but keep a person from functioning well or from feeling good. Many people with dysthymia also experience major depressive episodes at some time in their lives.

Affective disorders are often associated with a reduction in the central nervous system of certain biogenic amine neurotransmitters, such as dopamine, norepinephrine, and serotonin. Thus, many currently available treatments work primarily by raising biogenic amine neurotransmitter levels, by either inhibiting their uptake or preventing their metabolism. Affective disorders are commonly treated with antidepressant medications, including tricyclic antidepressants (TCAs), monoamine oxidase inhibitors (MAOIs), selective serotonin reuptake inhibitors (SSRIs), combined reuptake inhibitors and receptor blockers, serotonin and norepinephrine reuptake inhibitors (SNRIs), norepinephrine and dopamine reuptake inhibitors (NDRIs), and tetracyclic antidepressants.

The tricyclic antidepressants are “classical” antidepressants and include amitriptyline, amoxapine, desipramine (Norpramin®), doxepin (Sinequan®), imipramine (Tofranil®), nortriptyline (Pamelor®), protriptyline (Vivactil®), and trimipramine (Surmontil®). TCAs work by inhibiting the reuptake of serotonin and norepinephrine, and to a lesser extent, dopamine. However, they are not particularly selective with respect to the cell types they affect, and typically cause significant side effects, including drowsiness, dry mouth, blurred vision, constipation, urinary retention, dizziness, impaired sexual functioning, increased heart rate, disorientation or confusion, headache, low blood pressure, sensitivity to sunlight, increased appetite, weight gain, nausea, and weakness. TCAs may also increase instances of suicidal ideation. As a result of potential adverse reactions, TCAs are contraindicated in patients with heart disease (TCAs may precipitate a heart attack), narrow-angle glaucoma, thyroid problems, seizures, diabetes, or benign prostatic hypertrophy (BPH).

Monoamine oxidase inhibitors work as antidepressants by inhibiting the metabolism of serotonin, norepinephrine, and dopamine in order to maintain high levels of these neurotransmitters in the brain. MAOIs are associated with a wide range of serious side effects, including drowsiness, constipation, nausea, diarrhea, stomach upset, fatigue, dry mouth, dizziness, low blood pressure, lightheadedness (especially when getting up from a lying or sitting position), decreased urine output, decreased sexual function, sleep disturbances, muscle twitching, weight gain, blurred vision, headache, increased appetite, restlessness, shakiness, trembling, weakness, and increased sweating. MAOIs are also linked with increased suicidal ideation. In addition, MAOIs arouse serious safety concerns, and have been implicated in serotonin syndrome, a condition in which amounts of serotonin rise to life-threatening levels in the brain. Serotonin syndrome can occur if MAOIs are taken in combination with SSRIs. MAOIs also react with many food items, including cheeses, preserved foods, and alcoholic beverages. In the most serious cases, food interactions can lead to stroke in a patient. MAOIs may also cause serious drug-drug interactions with common medications, such as meperidine, and over-the-counter preparations, such as Saint John's Wort.

Selective serotonin reuptake inhibitors (SSRIs) were introduced as antidepressants in an attempt to limit some of the side effects of TCAs and MAOIs. SSRIs affect only serotonin in the brain, thus are more closely aligned with therapeutic heeds, and may attenuate unwanted side effects. Some SSRIs that are currently in use include citalopram (Celexa®), escitalopram (Lexapro®), fluoxetine (Prozac®, Prozac Weekly®), paroxetine (Paxil®, Paxil CR®), sertraline (Zoloft®). SSRIs are also associated with significant side effects, including nausea, sexual dysfunction (reduced desire or orgasm difficulties), headache, diarrhea, nervousness, rash, agitation, restlessness, increased sweating, weight gain, drowsiness, and insomnia. As with the other antidepressants, SSRIs are associated with increased suicidal ideation.

Combined reuptake inhibitors and receptor blockers were developed to not only block the reuptake of serotonin and norepinephrine, but also their receptors. Combined reuptake inhibitors and receptor blockers include trazodone, nefazodone, and maprotiline. Only generic forms of these drugs are sold currently because the brand name versions of these drugs are no longer available. Like other antidepressants, the combined reuptake inhibitors and receptor blockers cause the typical constellation of side effects, including dry mouth, dizziness, drowsiness, lightheadedness, nervousness, nausea, constipation, weakness, vision problems, confusion, and headache. Particular combined reuptake inhibitors and receptor blockers are associated with specific increased health risks. For example, trazadone is linked to priapism, sometimes requiring surgery and resulting in loss of erectile function or impotence. Nefazadone may in rare cases cause fatal liver failure. Maprotiline is linked to the onset of seizures. Combined reuptake inhibitors and receptor blockers may also increase suicidal ideation.

Serotonin and norepinephrine reuptake inhibitors include duloxetine (Cymbalta®) and venlafaxine (Effexor, Effexor XR®). Side effects of serotonin and norepinephrine reuptake inhibitors include nausea, vomiting, dizziness, insomnia, sleepiness, trouble sleeping, abnormal dreams, constipation, sweating, dry mouth, yawning, tremor, gas, anxiety, agitation, abnormal vision, such as blurred vision or double vision, headache, and sexual dysfunction. Both drugs increase the risk of serotonin syndrome if taken with certain drugs, such as MAOIs, or other over-the-counter preparations. Venlafaxine is linked to increased blood pressure and cholesterol. Both duloxitine and venlafaxine are contraindicated in patients with increased intraocular pressure or narrow-angle glaucoma. As with other antidepressants, duloxitine and venlafaxine may also increase suicidal ideation.

Norepinephrine and dopamine reuptake inhibitors (NDRIs) function by increasing norepinephrine and dopamine in the brain. Currently, bupropion (Wellbutrin®, Wellbutrin SR®, Wellbutrin XL®) is the only approved norepinephrine and dopamine reuptake inhibitor. Side effects of bupropion include loss of appetite, weight loss, headache, dry mouth, skin rash, sweating, ringing in the ears, shakiness and nervousness, stomach pain, agitation, constipation, anxiety, dizziness, trouble sleeping, muscle pain, nausea, and vomiting, fast heartbeat, sore throat, and more frequent urination. NDRIs are contraindicated in persons undergoing nicotine replacement therapy, and have been implicated in the onset of seizures and high blood pressure. Possible adverse drug or substance interactions may occur with MAOIs, alcohol, and certain sedatives. NDRIs may also increase suicidal ideation.

Tetracyclic antidepressants block binding at the alpha-2 receptor site, thus preventing the reabsorption of dopamine and serotonin in brain. Mirtazapine (Remeron®, Remeron SolTab®) is the only currently approved tetracyclic antidepressant. Side effects of mirtazapine include drowsiness, weight gain, dry mouth, dizziness, lightheadedness, thirst, muscle or joint aches, constipation, increased appetite, and increased cholesterol. Mirtazapine should not be taken with MAOIs, and, because it can cause drowsiness, should also not be taken with antihistamines, alcohol, or sedatives. Mirtazapine can in rare cases cause agranulocytosis, leading to increased vulnerability to infection. Patients should also be cautioned about the potential for suicidal ideation when taking mirtazapine.

The small molecule, ibudilast, (3-isobutyryl-2-isopropylpyrazolo[1,5-a]pyridine), is a non-selective inhibitor of cyclic nucleotide phosphodiesterase (PDE) (Fujimoto, T., et al., J. of Neuroimmunology, 95 (1999) 35-92). Ibudilast also acts as an LTD4 antagonist, an anti-inflammatory, a PAF antagonist, and a vasodilatatory agent (Thompson Current Drug Reports). Ibudilast is thought to exert a neuroprotective role in the central nervous system of mammals, presumably via suppression of the activation of glial cells (Mizuno et al. (2004) Neuropharmacology 46: 404-411). Ibudilast has been widely used in Japan for relieving symptoms associated with ischemic stroke or bronchial asthma. Marketed indications for ibudilast in Japan include its use as a vasodilator, for treating allergy, eye tissue regeneration, ocular disease, and treatment of allergic ophthalmic disease (Thompson Current Drug Reports). In recent clinical trials, its use in the treatment of multiple sclerosis, an inflammatory disease of the central nervous system, has been explored (News. Medical. Net; Pharmaceutical News, 2 Aug. 2005). While the use of ibudilast for a number of varying indications has been reported to date, to the best of the applicants' knowledge, its use in treating affective disorders has heretofore remained largely unexplored.

Currently available drugs for treating affective disorders unfortunately suffer from delayed onset of action, poor efficacy, and a variety of adverse side effects, as discussed above. Furthermore, a large number of individuals remain refractory to currently available therapies. In light of the shortcomings in current approaches, there exists a need for improved compositions and methods for treating affective disorders, particularly symptoms of depression, anxiety, and psychosis.

### SUMMARY OF THE INVENTION

In one aspect, the invention provides a method for treating an affective disorder, the method comprising administering to a subject in need thereof a therapeutically effective amount of ibudilast. Mammalian subjects suitable for treatment by the methods described herein include, but are not limited to, those suffering from an affective disorder with symptoms of depression, anxiety, or psychosis. In one embodiment, the subject is a human. In certain embodiments, ibudilast is administered systemically, for example, via intravenous, subcutaneous, intraperitoneal, oral, intranasal, sublingual or other systemic routes. In other embodiments, ibudilast is administered centrally, for example, intrathecally. In certain embodiments, multiple therapeutically effective doses of the ibudilast are administered to the subject.

A therapeutic dosage amount of ibudilast may be achieved by intermittent administration, or administration once daily (i.e., in a single dose), twice daily (i.e., in two separate doses), three times daily, or may be administered as multiple doses over a time course of several days, weeks, or even months. Such administering is typically over a duration of time effective to result in a diminution, and ideally elimination or even reversal, of symptoms of an affective disorder, such as depression, dysthymia, anxiety, or psychosis. Exemplary durations of treatment include at least about one week, from 1 week to 1 month, from two weeks to 2 months, up to about 6 months, up to about 12 months, 2 years, or even longer. In one particular embodiment, treatment lasts from about 1 week to about 50 weeks. In a preferred embodiment of the treatment method, the administering is over a duration of time effective to result in amelioration of symptoms of an affective disorder.

In certain embodiments, the method further comprises administering one or more other glial attenuators. Exemplary glial attenuators that may also be used in the practice of the invention include, but are not limited to, Minocycline, Fluorocitrate, MWO1-5-188WH, Propentofylline (also a PDE inhibitor), Pentoxyfylline (also a PDE inhibitor), Rolipram (also a PDE inhibitor), IL-10, IL-1 receptor antagonist(s), TNF-receptor antagonist(s) including sTNFR, MAP-kinase inhibitor(s), Yohimbine, glial cell chloride antagonists, caspase inhibitors, MMP inhibitors, cannabinoid receptor (e.g., type 2) agonists, arundic acid, statins, thalidomide and related analogs.

In certain embodiments, the method further comprises administering one or more other phosphodiesterase inhibitors. Exemplary phosphodiesterase inhibitors that may be used in the practice of the invention include, but are not limited to, PDE3, PDE4, PDE5, PDE7, and PDE10 inhibitors. For example, a PDE4 inhibitor can be selected from the group consisting of Rolipram, Arofylline, Doxofylline, Cipamfylline, Roflumilast, Tetomilast, Atizoram, CC-1088, Tofimilast, Tolafentrine, Pentoxyfylline, Dipyridamole, Cilostazol, Theophylline, Cilomilast, AWD-12-28, Propentofylline.

In certain embodiments, the method further comprises administering one or more other agents effective for treating an affective disorder, including but not limited to, an antidepressant, an antipsychotic, a tranquilizer, a sedative, a muscle relaxant, an anticonvulsant, and an insomnia therapeutic.

Exemplary antidepressants that may be used in the practice of the invention include, but are not limited to a tricyclic antidepressant (TCA), a monoamine oxidase inhibitor (MAOI), a selective serotonin reuptake inhibitor (SSRI), a combined reuptake inhibitor and receptor blocker, a serotonin and norepinephrine reuptake inhibitor (SNRI), a norepinephrine and dopamine reuptake inhibitor (NDRI), and a tetracyclic antidepressant.

In one embodiment, the sedative is a benzodiazepine. Exemplary benzodiazepines that may be used in the practice of the invention include, but are not limited to diazepam, chlordiazepoxide, alprazolam, clonazepam, temazepam, lorazepam, flurazepam, oxazepam, clorazepate and triazolam.

In another aspect, the invention provides a composition or combination effective for treating an affective disorder. The composition comprises ibudilast and optionally one or more additional agents effective for treating an affective disorder, wherein each of the components is either contained in a single composition or dosage form (such as in an admixture), or is present as a discrete or separate entity (e.g., in a kit). A composition of the invention may optionally include one or more pharmaceutically acceptable excipients.

In yet another aspect, the invention encompasses a kit comprising ibudilast, for the treatment of an affective disorder, such as depression, dysthymia, anxiety, or psychosis, and optionally, one or more additional agents effective for treating an affective disorder, for simultaneous, sequential or separate use. In certain embodiments, the one or more additional agents are selected from the group consisting of an antidepressant, an antipsychotic, a tranquilizer, a sedative, a muscle relaxant, an anticonvulsant, and an insomnia therapeutic.

These and other embodiments of the subject invention will readily occur to those of skill in the art in view of the disclosure herein.

### DETAILED DESCRIPTION OF THE INVENTION

The practice of the present invention will employ, unless otherwise indicated, conventional methods of chemistry, biochemistry, and pharmacology, within the skill of the art. Such techniques are explained fully in the literature. See, e.g.; A. L. Lehninger, Biochemistry (Worth Publishers, Inc., current addition); Morrison and Boyd, Organic Chemistry (Allyn and Bacon, Inc., current addition); J. March, Advanced Organic Chemistry (McGraw Hill, current addition); Remington: The Science and Practice of Pharmacy, A. Gennaro, Ed., 20^{th} Ed.; Goodman & Gilman The Pharmacological Basis of Therapeutics, J. Griffith Hardman, L. L. Limbird, A. Gilman, 10^{th} Ed.

All publications, patents and patent applications cited herein, whether supra or infra, are hereby incorporated by reference in their entirety.

### I. Definitions

In describing and claiming the present invention, the following terminology will be used in accordance with the definitions described below.

It must be noted that, as used in this specification and the intended claims, the singular forms “a,” “an,” and “the” include plural referents unless the context clearly dictates otherwise. Thus, for example, reference to “a drug” includes a single drug as well as two or more of the same or different drugs, reference to “an optional excipient” refers to a single optional excipient as well as two or more of the same or different optional excipients, and the like.

The term “affective disorder” refers to any type of mood disorder with symptoms including, but not limited to, depression, anxiety, and/or psychosis. These disorders are characterized by various symptoms including, but not limited to interference with the ability to work, study, sleep, eat, and enjoy once pleasurable activities. Additional symptoms of depression may include persistent sadness, anxiety, or “empty” mood, feelings of hopelessness, pessimism, guilt, worthlessness, or helplessness, loss of interest or pleasure in hobbies and activities that were once enjoyed, including sex, decreased energy, fatigue, and a sense of being “slowed down,” restlessness, irritability, difficulty concentrating, remembering, or making decisions, sleep disturbances, such as insomnia, early-morning awakening, or oversleeping, loss of appetite and/or weight loss or overeating and weight gain, thoughts of death or suicide and/or suicide attempts, and persistent physical symptoms that do not respond to treatment, such as headaches, digestive disorders, and chronic pain.

These disorders are well documented and diagnosis of these disorders is typically accomplished by mental health providers using the Diagnostic and Statistical Manual of Mental Disorders (DSM), published by the American Psychiatric Association, Washington D.C.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Active molecule” or “active agent” as described herein includes any agent, drug, compound, composition of matter or mixture which provides some pharmacologic, often beneficial, effect that can be demonstrated in-vivo or in vitro. This includes foods, food supplements, nutrients, nutriceuticals, drugs, vaccines, antibodies, vitamins, and other beneficial agents. As used herein, the terms further include any physiologically or pharmacologically active substance that produces a localized or systemic effect in a patient.

“Substantially” or “essentially” means nearly totally or completely, for instance, 95% or greater of some given quantity.

“Optional” or “optionally” means that the subsequently described circumstance may or may not occur, so that the description includes instances where the circumstance occurs and instances where it does not.

The term “central nervous system” or “CNS” includes all cells and tissue of the brain and spinal cord of a vertebrate. Thus, the term includes, but is not limited to, neuronal cells, glial cells (astrocytes, microglia, oligodendrocytes), cerebrospinal fluid (CSF), interstitial spaces and the like.

“Glial cells” refer to various cells of the CNS also known as microglia, astrocytes, and oligodendrocytes.

The terms “subject”, “individual” or “patient” are used interchangeably herein and refer to a vertebrate, preferably a mammal. Mammals include, but are not limited to, murines, rodents, simians, humans, farm animals, sport animals and pets.

The term “about”, particularly in reference to a given quantity, is meant to encompass deviations of plus or minus five percent.

The terms “effective amount” or “pharmaceutically effective amount” of a composition or agent, as provided herein, refer to a nontoxic but sufficient amount of the composition to provide the desired response, such as suppression of glial activation, inhibition of phosphodiesterase activity, or inhibition of leukotriene D4 synthesis in a subject, and optionally, a corresponding therapeutic effect, such as preventing, diminishing, or eliminating symptoms of an affective disorder (e.g., depression, dysthymia, anxiety, or psychosis) in a subject. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation.

By “therapeutically effective dose or amount” of ibudilast is intended an amount that, when ibudilast is administered as described herein, brings about a positive therapeutic response in treatment of an affective disorder, such as preventing, diminishing, or eliminating symptoms of depression, dysthymia, anxiety, or psychosis in a subject.

### II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that ibudilast can be used for treating affective disorders, particularly symptoms of depression, psychosis, and anxiety. Although not wishing to be bound to any particular theory, the following properties of ibudilast may contribute to its efficacy in treating depression, psychosis, and anxiety: 1) ibudilast partitions to the central nervous system, 2) targets some of the underlying pathophysiology associated with depression, psychosis, and anxiety (e.g., suppresses inflammation and glial cell activation in the central nervous system), 3) and exhibits desired pharmacological activity at well tolerated doses in humans and animals (e.g., inhibits phosphodiesterase activity, including PDE3, PDE4, PDE5, PDE7, and PDE10, and leukotriene D4 synthesis). This constellation of demonstrated properties sets ibudilast apart as a unique potential therapy for depression, psychosis, and anxiety.

The drugs presently used to treat depressive illness, have multiple undesirable side-effects, are not effective for all patients, do not provide relief for all symptoms, and have many serious potential safety problems. Ibudilast, as a glial attenuator, non-specific PDE inhibitor, and a leukotriene D4 inhibitor, works by an entirely different mechanism than all other antidepressants.

As an attenuator of microglial activation, ibudilast can effectively treat depressive illness by suppressing glial activation in the central nervous system. Pro-inflammatory cytokines, which are induced as a result of microglial activation within the central nervous system, have been implicated in the development of pathological depression (Strouse (2007) Current Pain and Headache Reports 11:98-103; Illman et al. (2005) J. Support. Oncol. 3:37-50). The induction of proinflammatory cytokines in response to glial activation results in depletion of serum levels of tryptophan, a precursor for serotonin synthesis. Tryptophan depletion has been studied in humans and shown to induce depressive relapse in medically well patients who suffer from depressive disorders (Delgado et al. (2004) Arch. Genet. Psychol. 51:865-874). In addition to depression, pro-inflammatory cytokines also elicit chronic pain resulting in the frequent comorbidity of depression and neuropathic pain in patients.

The inhibition of certain phosphodiesterases has been extensively explored and linked to potential human therapeutic utility for treating depression, cognition, and psychosis (Menniti et al. (2006) Nature Reviews; Drug Discovery 5:660-670). Notably, ibudilast has inhibitory activity against PDE's 3, 4, 5, 7, and 10, which have been linked to depression and psychosis.

PDE4 has shown potential as a candidate target for therapy of depression (Bender et al. (2006) Pharmacol. Rev. 58:488-520). Inhibitors of PDE4 have exhibited antidepressant effects possibly mediated in part by upregulation of brain derived neurotrophic factor (Menniti et al. (2006), supra).

PDE10 inhibition, in particular, is linked to utility in treating psychosis (Menniti et al. (2007) Curr. Opin. Investig. Drugs 8:54-59). Deletion or inhibition of PDE10 reduces conditioned avoidance responses. Ibudilast is a potent PDE10 inhibitor with Kᵢ values for PDE10 isozymes ranging from 1.3 to 2.2 mM (L. C. D. Gibson Eur J. Pharmacol. 538:39-42, 2006).

PDE3 antagonizes cAMP activity, and cAMP has been shown to enhance glucocorticoid receptor function and inhibit pro-inflammatory cytokine signaling. A number of neuropsychiatric disorders, including major depression, are associated with decreased responsiveness to glucocorticoids (Pace et al. (2007) Brain Behav. Immun. 21:9-19). Glucocorticoid resistance may result from impaired glucocorticoid receptor function related to chronic exposure to inflammatory cytokines as may occur during chronic medical illness or chronic stress. Indeed, inflammatory cytokines and their signaling pathways including mitogen-activated protein kinases, nuclear factor-kappaB, signal transducers and activators of transcription, and cyclooxygenase have been found to inhibit glucocorticoid receptor function. Without being bound by a particular theory, the effectiveness of ibudilast in treating neuropsychiatric disorders, such as depression, may be mediated in part through inhibition of PDE3, which results in increased levels of circulating cAMP, inhibition of proinflammatory cytokine activity, and enhanced glucocorticoid receptor-function.

Additionally, the inhibition of leukotriene D4 synthesis by ibudilast (Huang et al. (2006) Life Sci. 78:2663-2668) may be effective for treating depressive illness and anxiety (Manev et al. (2004) Crit. Rev. Neurobiol. 16:181-186; Manev et al. (2007) Medical Hypotheses (In Press).

In order to further an understanding of the invention, a more detailed discussion is provided below regarding methods of treating affective disorders with ibudilast, particularly symptoms of depression, psychosis, and anxiety.

Treatment of Affective Disorders with Ibudilast

The invention relates to the use of ibudilast for treating affective disorders. Ibudilast is believed to ameliorate symptoms of depression, psychosis, and anxiety.

In certain embodiments, the invention provides a method for treating an affective disorder, comprising administering to a subject in need thereof a therapeutically effective amount of ibudilast. Mammalian subjects suitable for treatment by the methods described herein include, but are not limited to, those suffering from an affective disorder including symptoms of depression, psychosis, and/or anxiety. Such disorders include, but are not limited to, depression, bipolar disorder, post-partum depression, dysthymia, seasonal affective disorder, schizoaffective disorder, general anxiety disorder, panic disorder, and posttraumatic stress disorder.

In certain embodiments, the method further comprises administering one or more other glial attenuators. Exemplary glial attenuators that may also be used in the practice of the invention include, but are not limited to, Minocycline, Fluorocitrate, MWO1-5-188WH, Propentofylline (also a PDE inhibitor), Pentoxyfylline (also a PDE inhibitor), Rolipram (also a PDE inhibitor), IL-10, IL-1 receptor antagonist(s), TNF-receptor antagonist(s) including sTNFR, MAP-kinase inhibitor(s), Yohimbine, glial cell chloride antagonists, caspase inhibitors, MMP inhibitors, cannabinoid receptor (e.g., type 2) agonists, arundic acid, statins, thalidomide and related analogs.

In certain embodiments, the method further comprises administering one or more other phosphodiesterase inhibitors. Exemplary phosphodiesterase inhibitors that may be used in the practice of the invention include, but are not limited to, PDE3, PDE4, PDE5, PDE7, and PDE10 inhibitors. For example, a PDE4 inhibitor can be selected from the group consisting of Rolipram, Arofylline, Doxofylline, Cipamfylline, Roflumilast, Tetomilast, Atizoram, CC-1088, Tofimilast, Tolafentrine, Pentoxyfylline, Dipyridamole, Cilostazol, Theophylline, Cilomilast, AWD-12-28, Propentofylline.

In certain embodiments, a therapeutically effective amount of ibudilast is administered to a subject in combination therapy with one or more other agents for treating an affective disorder. Such agents include, but are not limited to, antidepressants, antipsychotics, tranquilizers, sedatives, muscle relaxants, anticonvulsants, and insomnia therapeutics.

Exemplary antidepressants that may be used in the practice of the invention include, but are not limited to a tricyclic antidepressant, such as amitriptyline, amoxapine, desipramine (Norpramin®), doxepin (Sinequan®), imipramine (Tofranil®), nortriptyline (Pamelor®), protriptyline (Vivactil®), and trimipramine (Surmontil®); a monoamine oxidase inhibitor, such as isocarboxazid, pargyline, selegiline, furazolidone and phenelzine; a selective serotonin reuptake inhibitor, such as citalopram (Celexa®), escitalopram (Lexapro®), fluoxetine (Prozac®, Prozac Weekly®), paroxetine (Paxil®, Paxil CR®), sertraline (Zoloft®); a combined reuptake inhibitor and receptor blocker, such as trazodone, nefazodone, and maprotiline; a serotonin and norepinephrine reuptake inhibitor, such as duloxetine (Cymbalta®) and venlafaxine (Effexor, Effexor XR®); a norepinephrine and dopamine reuptake inhibitor, such as bupropion (Wellbutrin®, Wellbutrin SR®, Welibutrin XL®); and a tetracyclic antidepressant, such as Mirtazapine (Remeron®, Remeron SolTab®).

In one embodiment, the sedative is a benzodiazepine. Exemplary benzodiazepines that may be used in the practice of the invention include, but are not limited to diazepam, chlordiazepoxide, alprazolam, clonazepam, temazepam, lorazepam, flurazepam, oxazepam, clorazepate and triazolam.

Exemplary insomnia therapeutics that may be used in the practice of the invention include, but are not limited to flurazepam, temazepam, zolpidem tartrate, eszopiclone, diphenhydramine, doxylamine.

### Pharmaceutical Compositions

Ibudilast

Ibudilast is a small molecule drug (molecular weight of 230.3) having the structure shown below.

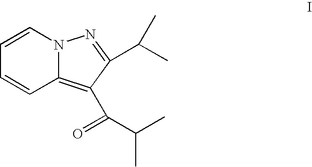

Ibudilast is also found under ChemBank ID 3227, CAS # 50847-11-5, and Beilstein Handbook Reference No. 5-24-03-00396. Its molecular formula corresponds to [C₁₄H₁₈N₂O]. Ibudilast is also known by various chemical names which include 2-methyl-1-(2-(1-methylethyl)pyrazolo(1,5-a)pyridin-3-yl) 1-propanone; 3-isobutyryl-2-isopropylpyrazolo(1,5-a)pyridine]; and 1-(2-isopropyl-pyrazolo[1,5-a]pyridin-3-yl)-2-methyl-propan-1-one. Other synonyms for ibudilast include Ibudilastum (Latin), BRN 0656579, KC-404, and the brand name Ketas®. Ibudilast, as referred to herein, is meant to include any and all pharmaceutically acceptable salt forms thereof, prodrug forms (e.g., the corresponding ketal), and the like, as appropriate for use in its intended formulation for administration.

Ibudilast is a non-selective nucleotide phosphodiesterase (PDE) inhibitor (most active against PDE-3, PDE-4, PDE-10, and PDE-11 (Gibson et al. (2006) Eur. J. Pharmacology 538:39-42)), and has also been reported to have LTD4 and PAF antagonistic activities. Its profile appears effectively anti-inflammatory and unique in comparison to other PDE inhibitors and anti-inflammatory agents. PDEs catalyze the hydrolysis of the phosphoester bond on the 3′-carbon to yield the corresponding 5′-nucleotide monophosphate. Thus, they regulate the cellular concentrations of cyclic nucleotides. Since extracellular receptors for many hormones and neurotransmitters utilize cyclic nucleotides as second messengers, the PDEs also regulate cellular responses to these extracellular signals. There are 11 families of PDES: Ca²⁺/calmodulin-dependent PDEs (PDE1); cGMP-stimulated PDEs (PDE2); cGMP-inhibited PDEs (PDE3); cAMP-specific PDEs (PDE4); cGMP-binding PDEs (PDE5); photoreceptor PDEs (PDE6); high affinity, cAMP-specific PDEs (PDE7); specific PDE (PDE8); high affinity cGMP-specific PDEs (PDE9); and mixed cAMP and cGMP PDEs (PDE10, PDE11).

As stated previously, a reference to any one or more of the herein-described drugs, in particular ibudilast, is meant to encompass, where applicable, any and all enantiomers, mixtures of enantiomers including racemic-mixtures, prodrugs, pharmaceutically acceptable salt forms, hydrates (e.g., monohydrates, dihydrates, etc.), different physical forms (e.g., crystalline solids, amorphous solids), metabolites, and the like.

Formulation Components

In addition to comprising ibudilast, the compositions of the invention may optionally contain one or more additional components as described below.

Excipients/Carriers

In addition to ibudilast, the compositions of the invention for treating an affective disorder may further comprise one or more pharmaceutically acceptable excipients or carriers. Exemplary excipients include, without limitation, polyethylene glycol (PEG), hydrogenated castor oil (HCO), cremophors, carbohydrates, starches (e.g., corn starch), inorganic salts, antimicrobial agents, antioxidants, binders/fillers, surfactants, lubricants (e.g., calcium or magnesium stearate), glidants such as talc, disintegrants, diluents, buffers, acids, bases, film coats, combinations thereof, and the like.

A composition of the invention may include one or more carbohydrates such as a sugar, a derivatized sugar such as an alditol, aldonic acid, an esterified sugar, and/or a sugar polymer. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

Also suitable for use in the compositions of the invention are potato and corn-based starches such as sodium starch glycolate and directly compressible modified starch.

Further representative excipients include inorganic salt or buffers such as citric acid, sodium chloride, potassium chloride, sodium sulfate, potassium nitrate, sodium phosphate monobasic, sodium phosphate dibasic, and combinations thereof.

A composition comprising ibudilast may also include an antimicrobial agent, e.g., for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

A composition comprising ibudilast may also contain one or more antioxidants. Antioxidants are used to prevent oxidation, thereby preventing the deterioration of the drug(s) or other components of the preparation. Suitable antioxidants for use in the present, invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

Additional excipients include surfactants such as polysorbates, e.g., “Tween 20” and “Tween 80,” and pluronics such as F68 and F88 (both of which are available from BASF, Mount Olive, N.J.), sorbitan esters, lipids (e.g., phospholipids such as lecithin and other phosphatidylcholines, and phosphatidylethanolamines), fatty acids and fatty esters, steroids such as cholesterol, and chelating agents, such as EDTA, zinc and other such suitable cations.

Further, a composition comprising ibudilast may optionally include one or more acids or bases. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumarate, and combinations thereof.

The amount, of any individual excipient in the composition will vary depending on the role of the excipient, the dosage requirements of the active agent components, and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient. In general, the amount of excipient present in a 3,4,6-substituted pyridazine composition is selected from the following: at least about 2%, 5%, 10%, 15%, 20%, 25%, 30%, 35%, 40%, 45%, 50%, 55%, 60%, 65%, 70%, 75%, 80%, 85%, 90%, or even 95% by weight.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

Other Actives

As described above, the formulation (or kit) in accordance with the invention may contain, in addition to ibudilast, one or more other glial attenuators, phosphodiesterase inhibitors (e.g., PDE3, PDE4, PDE5, PDE7, and PDE10 inhibitors), leukotriene D4 synthesis inhibitors, or other agents effective for treating affective disorders, including but not limited to, antidepressants, antipsychotics, tranquilizers, sedatives, muscle relaxants, anticonvulsants, and insomnia therapeutics. Such actives include glial attenuators, such as Minocycline, Fluorocitrate, MWO 1-5-188WH, Propentofylline (also a PDE inhibitor), Pentoxyfylline (also a PDE inhibitor), Rolipram (also a PDE inhibitor), IL-10, IL-1 receptor antagonist(s), TNF-receptor antagonist(s) including sTNFR, MAP-kinase inhibitor(s), Yohimbine, glial cell chloride antagonists, caspase inhibitors, MMP inhibitors, cannabinoid receptor (e.g., type 2) agonists, arundic acid, statins, thalidomide and related analogs; phosphodiesterase inhibitors, such as Rolipram, Arofylline, Doxofylline, Cipamfylline, Roflumilast, Tetomilast, Atizoram, CC-1088, Tofimilast, Tolafentrine, Pentoxyfylline, Dipyridamole, Cilostazol, Theophylline, Cilomilast, AWE-12-28, Propentofylline; antidepressants, such as tricyclic antidepressants, including but not limited to, amitriptyline, amoxapine, desipramine (Norpramin®), doxepin (Sinequan®), imipramine (Tofranil®), nortriptyline (Pamelor®), protriptyline (Vivactil®), and trimipramine (Surmontil®); monoamine oxidase inhibitors, including but not limited to, isocarboxazid, pargyline, selegiline, furazolidone and phenelzine; selective serotonin reuptake inhibitors, including but not limited to, citalopram (Celexa®), escitalopram (Lexapro®), fluoxetine (Prozac®, Prozac Weekly®), paroxetine (Paxil®, Paxil CR®), sertraline (Zoloft®); combined reuptake inhibitors and receptor blockers, including but not limited to, trazodone, nefazodone, and maprotiline; serotonin and norepinephrine reuptake inhibitors, including but not limited to, duloxetine (Cymbalta®) and venlafaxine (Effexor, Effexor XR®); norepinephrine and dopamine reuptake inhibitors, including but not limited to, bupropion (Wellbutrin®, Wellbutrin SR®, Wellbutrin XL®); and tetracyclic antidepressants, including but not limited to, Mirtazapine (Remeron®, Remeron SolTab®); benzodiazepines, such as diazepam, chlordiazepoxide, alprazolam, clonazepam, temazepam, lorazepam, flurazepam, oxazepam, clorazepate and triazolam; insomnia therapeutics, such as flurazepam, temazepam, zolpidem tartrate, eszopiclone, diphenhydramine, and doxylamine.

The precise amount of a secondary active agent to be administered during combination therapy with ibudilast will, of course, be adjusted accordingly and will depend upon factors such as intended patient population, the particular affective disorder symptom or condition to be treated, potential synergies between the active agents administered, and the like, and will readily be determined by one skilled in the art based upon the guidance provided herein.

Sustained Delivery Formulations

The compositions may also be formulated in order to improve stability and extend the half-life of ibudilast. For example, ibudilast may be delivered in a sustained-release formulation. Controlled or sustained-release formulations are prepared by incorporating ibudilast into a carrier or vehicle such as liposomes, nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures. Additionally, ibudilast can be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996).

### Delivery Forms

The compositions comprising ibudilast described herein encompass all types of formulations, and in particular, those that are suited for systemic or intrathecal administration. Oral dosage forms include tablets, lozenges, capsules, syrups, oral suspensions, emulsions, granules, and pellets. Alternative formulations include aerosols, transdermal patches, gels, creams, ointments, suppositories, powders or lyophilates that can be reconstituted, as well as liquids. Examples of suitable diluents for reconstituting solid compositions, e.g., prior to injection, include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned. Preferably, a composition comprising ibudilast is one suited for oral administration.

In turning now to oral delivery formulations, tablets can be made by compression or molding, optionally with one or more accessory ingredients or additives. Compressed tablets are prepared, for example, by compressing in a suitable tabletting machine, the active ingredients in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) and/or surface-active or dispersing agent.

Molded tablets are made, for example, by molding in a suitable tabletting machine, a mixture of powdered compounds moistened with an inert liquid diluent. The tablets may optionally be coated or scored, and may be formulated so as to provide slow or controlled release of the active ingredients, using, for example, hydroxypropylmethyl cellulose in varying proportions to provide the desired release profile. Tablets may optionally be provided with a coating, such as a thin film, sugar coating, or an enteric coating to provide release in parts of the gut other than the stomach. Processes, equipment, and toll manufacturers for tablet and capsule making are well-known in the art.

Formulations for topical administration in the mouth include lozenges comprising the active ingredients, generally in a flavored base such as sucrose and acacia or tragacanth and pastilles comprising the active ingredients in an inert base such as gelatin and glycerin or sucrose and acacia.

A pharmaceutical composition for topical administration may also be formulated as an ointment, cream, suspension, lotion, powder, solution, paste, gel, spray, aerosol or oil.

Alternatively, the formulation may be in the form of a patch (e.g., a transdermal patch) or a dressing such as a bandage or adhesive plaster impregnated with active ingredients and optionally one or more excipients or diluents. Topical formulations may additionally include a compound that enhances absorption or penetration of the ingredients through the skin or other affected areas, such as dimethylsulfoxidem bisabolol, oleic acid, isopropyl myristate, and D-limonene, to name a few.

For emulsions, the oily phase is constituted from known ingredients in a known manner. While this phase may comprise merely an emulsifier (otherwise known as an emulgent), it desirably comprises a mixture of at least one emulsifier with a fat and/or an oil. Preferably, a hydrophilic emulsifier is included together with a lipophilic emulsifier that acts as a stabilizer. Together, the emulsifier(s) with or without stabilizer(s) make up the so-called emulsifying wax, and the wax together with the oil and/or fat make up the so-called emulsifying ointment base which forms the oily dispersed phase of cream formulations. Illustrative emulgents and emulsion stabilizers include Tween 60, Span 80, cetostearyl alcohol, myristyl alcohol, glyceryl monostearate and sodium lauryl sulfate.

Formulations for rectal administration are typically in the form of a suppository with a suitable base comprising, for example, cocoa butter or a salicylate.

Formulations suitable for vaginal administration generally take the form of a suppository, tampon, cream, gel, paste, foam or spray.

Formulations suitable for nasal administration, wherein the carrier is a solid, include a coarse powder having a particle size, for example, in the range of about 20 to about 500 microns. Such a formulation is typically administered by rapid inhalation through the nasal passage, e.g., from a container of the powder held in proximity to the nose. Alternatively, a formulation for nasal delivery may be in the form of a liquid, e.g., a nasal spray or nasal drops.

Aerosolizable formulations for inhalation may be in dry powder form (e.g., suitable for administration by a dry powder inhaler), or, alternatively, may be in liquid form, e.g., for use in a nebulizer. Nebulizers for delivering an aerosolized solution include the AERX™ (Aradigm), the Ultravent® (Mallinkrodt), and the Acorn II® (Marquest Medical Products). A composition of the invention may also be delivered using a pressurized, metered dose inhaler (MDI), e.g., the Ventolin® metered dose inhaler, containing a solution or suspension of a combination of drugs as described herein in a pharmaceutically inert liquid propellant, e.g., a chlorofluorocarbon or fluorocarbon.

Formulations suitable for parenteral administration include aqueous and non-aqueous isotonic sterile solutions suitable for injection, as well as aqueous and non-aqueous sterile suspensions.

Parenteral formulations are optionally contained in unit-dose or multi-dose sealed containers, for example, ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the types previously described.

A formulation for use with the invention may also be a sustained release formulation, such that each of the drug components is released or absorbed slowly over time, when compared to a non-sustained release formulation. Sustained release formulations may employ pro-drug forms of the active agent, delayed-release drug delivery systems such as liposomes or polymer matrices, hydrogels, or covalent attachment of a polymer such as polyethylene glycol to the active agent.

In addition to the ingredients particularly mentioned above, the formulations may optionally include other agents conventional in the pharmaceutical arts and particular type of formulation being employed, for example, for oral administration forms, the composition for oral administration may also include additional agents as sweeteners, thickeners or flavoring agents.

The compositions comprising ibudilast may also be prepared in a form suitable for veterinary applications.

### Kits

Also provided herein is a kit containing a composition comprising ibudilast accompanied by instructions for use, e.g., in treating an affective disorder, particularly symptoms of depression, anxiety, or psychosis. In addition, the kit may optionally contain one or more other glial attenuators, phosphodiesterase inhibitors (e.g., PDE3, PDE4, PDE5, PDE7, and PDE10 inhibitors), leukotriene D4 synthesis inhibitors, or other agents effective for treating affective disorders, including but not limited to, antidepressants, antipsychotics, tranquilizers, sedatives, muscle relaxants, anticonvulsants, and insomnia therapeutics.

For example, in instances in which each of the drugs themselves are administered as individual or separate dosage forms, the kit comprises ibudilast in addition to each of the drugs making up the composition of the invention, along with instructions for use. Ibudilast and one or more other agents may be present in the same or separate compositions. The drug components may be packaged in any manner suitable for administration, so long as the packaging, when considered along with the instructions for administration, clearly indicates the manner in which each of the drug components is to be administered.

For example, for an illustrative kit comprising ibudilast and a sedative, the kit may be organized by any appropriate time period, such as by day. As an example, for Day 1, a representative kit may comprise unit dosages of each of ibudilast and the sedative. If each of the drugs is to be administered twice daily, then the kit may contain, corresponding to Day 1, two rows of unit dosage forms of each of ibudilast and the sedative, along with instructions for the timing of administration. Alternatively, if one or more of the drugs differs in the timing or quantity of unit dosage form to be administered in comparison to the other drug members of the combination, then such would be reflected in the packaging and instructions. Various embodiments according to the above may be readily envisioned, and would of course depend upon the particular combination of drugs, in addition to ibudilast, employed for treatment, their corresponding dosage forms, recommended dosages, intended patient population, and the like. The packaging may be in any form commonly employed for the packaging of pharmaceuticals, and may utilize any of a number of features such as different colors, wrapping, tamper-resistant packaging, blister packs, dessicants, and the like.

### Method of Administration

As set forth above, the present invention encompasses a method of treating an affective disorder, particularly symptoms of depression, anxiety, or psychosis, by administering a therapeutically effective dosage of ibudilast. Such administering is effective to ameliorate symptoms of the affective disorder experienced by the subject, i.e., to result in significant attenuation or elimination of depression, anxiety, or psychosis.

Therapeutic amounts can be empirically determined and will vary with the particular condition being treated, the subject, and the particular efficacy and toxicity of each of the active agents contained in the composition. The actual dose to be administered will vary depending upon the age, weight, and general condition of the subject as well as the severity of the condition being treated, the judgment of the health care professional, and particular mode of administration.

The method of the invention may, in certain instances, comprise a step of selecting a subject experiencing depression, anxiety, or psychosis prior to administering thereto ibudilast. Such subjects are typically selected from those suffering from an affective disorder, including but not limited to, depression, bipolar disorder, postpartum depression, dysthymia, seasonal affective disorder, schizoaffective disorder, general anxiety disorder, panic disorder, and posttraumatic stress disorder.

Ibudilast may also be administered in combination with one or more other glial attenuators, phosphodiesterase inhibitors (e.g., PDE3, PDE4, PDE5, PDE7, and PDE10 inhibitors), leukotriene D4 synthesis inhibitors, or other agents effective for treating affective disorders. Exemplary agents include, but are not limited to, antidepressants, antipsychotics, tranquilizers, sedatives, muscle relaxants, anticonvulsants, and insomnia therapeutics.

Preferred methods of delivery of therapeutic formulations comprising ibudilast for the treatment of an affective disorder include systemic and localized delivery, i.e., directly into the central nervous system. Such routes of administration include, but are not limited to, oral, intra-arterial, intrathecal, intraspinal, intramuscular, subcutaneous, intraperitoneal, intravenous, intranasal, and inhalation routes.

More particularly, a formulation containing ibudilast of the present invention may be administered for therapy by any suitable route, including without limitation, oral, rectal, nasal, topical (including transdermal, aerosol, buccal and sublingual), vaginal, parenteral (including subcutaneous, intramuscular, intravenous and intradermal), intrathecal, and pulmonary. The preferred route will, of course, vary with the condition and age of the recipient, the particular affective disorder being treated, and the specific combination of drugs employed.

One preferred mode of administration for delivery of ibudilast is directly to neural tissue such as peripheral nerves, the retina, dorsal root ganglia, neuromuscular junction, as well as the CNS, e.g., to target spinal cord glial cells by injection into, e.g., the ventricular region, as well as to the striatum (e.g., the caudate nucleus or putamen of the striatum), spinal cord and neuromuscular junction, with a needle, catheter or related device, using neurosurgical techniques known in the art, such as by stereotactic injection (see, e.g., Stein et al., J. Virol. 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

A particularly preferred method for targeting spinal cord glia is by intrathecal delivery, rather than into the cord tissue itself.

Another preferred method for administering the compositions comprising ibudilast of the invention is by delivery to dorsal root ganglia (DRG) neurons, e.g., by injection into the epidural space with subsequent diffusion to DRG. For example, an ibudilast-based composition can be delivered via intrathecal cannulation under conditions where ibudilast is diffused to DRG. See, e.g., Chiang et al., Acta Anaesthesiol. Sin. (2000) 38:31-36; Jain, K. K., Expert Opin. Investig. Drugs (2000) 9:2403-2410.

Yet another mode of administration to the CNS uses a convection-enhanced delivery (CED) system. In this way, ibudilast can be delivered to many cells over large areas of the CNS. Any convection-enhanced delivery device may be appropriate for delivery of ibudilast. In a preferred embodiment, the device is an osmotic pump or an infusion pump. Both osmotic and infusion pumps are commercially available from a variety of suppliers, for example Alzet Corporation, Hamilton Corporation, Alza, Inc., Palo Alto, Calif.). Typically, a composition comprising ibudilast of the invention is delivered via CED devices as follows. A catheter, cannula or other injection device is inserted into CNS tissue in the chosen subject. Stereotactic maps and positioning devices are available, for example from ASI Instruments, Warren, Mich. Positioning may also be conducted by using anatomical maps obtained by CT and/or MRI imaging to help guide the injection device to the chosen target. For a detailed description regarding CED delivery, see U.S. Pat. No. 6,309,634, incorporated herein by reference in its entirety.

A composition comprising ibudilast, when comprising more than one active agent, may be administered as a single combination composition comprising a combination of ibudilast and at least one additional active agent effective in the treatment of an affective disorder. In terms of patient compliance and ease of administration, such an approach is preferred, since patients are often adverse to taking multiple pills or dosage forms, often multiple times daily, over the duration of treatment. Alternatively, albeit less preferably, the combination of the invention is administered as separate dosage forms. In instances in which the drugs comprising the therapeutic composition of the invention are administered as separate dosage forms and co-administration is required, ibudilast and each of the additional active agents may be administered simultaneously, sequentially in any order, or separately.

Dosages

Therapeutic amounts can be empirically determined by those skilled in the art and will vary with the particular condition being treated, the subject, and the efficacy and toxicity of each of the active agents contained in the composition. The actual dose to be administered will vary depending upon the age, weight, and general condition of the subject as well as the severity of the condition being treated, the judgment of the health care professional, and the particular combination of ibudilast, and any other agents being administered.

Therapeutically effective amounts can be determined by those skilled in the art, and will be adjusted to the requirements of each particular case. Generally, a therapeutically effective amount of ibudilast will range from a total daily dosage, for example in humans, of about 0.1 and 500 mg/day, more preferably, in an amount between 1 and 200 mg/day, 1 and 100 mg/day, 1 and 40 mg/day, or 1 and 20 mg/day, administered as either a single dosage or as multiple dosages.

Depending upon the dosage amount and precise condition to be treated, administration can be one, two, or three times daily, or even more, for a time course of one day to several days, weeks, months, and even years, and may even be for the life of the patient. Intermittent dosing may also be employed, e.g., to treat symptoms of depression, anxiety, or psychosis, with a maximal dose not to be exceeded as recommended by the practicing physician. Illustrative dosing regimes will last a period of at least about a week, from about 1-4 weeks, from 1-3 months, from 1-6 months, from 1-50 weeks, from 1-12 months, from 1-2 years, or longer.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Materials and Methods

Ibudilast is obtained as a pure powder from Sigma (St. Louis, Mo.) or Haorui Pharma (Edison, N.J.) and prepared daily as a solution for intraperitoneal or oral administration. Previous range-finding tolerability and efficacy studies in neurological models indicate that ibudilast is well-tolerated intraperitoneally at dose levels up to 15 mg/kg twice a day (bid) for multiple days. An appropriate amount of ibudilast is dissolved in 100% polyethylene (PEG) 400 (Sigma) and then diluted down to a final concentration of 35% PEG400 in sterile saline (0.9% for injection). Drug stability and concentration are validated by HPLC/MS/MS.

Thus, the use of ibudilast to treat affective disorders is described. In particular, ibudilast can be used to treat symptoms of depression, psychosis, or anxiety. Although preferred embodiments of the subject invention have been described in some detail, it is understood that obvious variations can be made without departing from the spirit and the scope of the invention as claimed herein.

All references cited herein, including patents, patent applications and other publications, are hereby incorporated by reference in their entireties.

What is claimed is:

1. A method for treating an affective disorder, the method comprising administering to a subject in need thereof a therapeutically effective amount of ibudilast.

2. The method of claim 1, wherein the affective disorder is depression.

3. The method of claim 1, wherein the affective disorder is dysthymia

4. The method of claim 1, wherein the affective disorder is anxiety.

5. The method of claim 1, wherein the affective disorder is psychosis.

6. The method of claim 1, wherein the subject is human.

7. The method of claim 1, wherein the ibudilast is administered systemically.

8. The method of claim 7, wherein the ibudilast is administered intravenously, subcutaneously, intraperitoneally, orally, intranasally, or sublingually.

9. The method of claim 8, wherein the ibudilast is administered orally.

10. The method of claim 8, wherein the ibudilast is administered intraperitoneally.

11. The method of claim 1, wherein the ibudilast is administered centrally.

12. The method of claim 11, wherein the ibudilast is administered intrathecally.

13. The method of claim 1, wherein multiple therapeutically effective doses of the ibudilast are administered to the subject.

14. The method of claim 1, further comprising administering one or more other glial attenuators.

15. The method of claim 14, wherein the one or more glial attenuators is selected from the group consisting of Minocycline, Fluorocitrate, MWO1-5-188WH, Propentofylline, Pentoxyfylline, Rolipram, IL-10, an IL-1 receptor antagonist, a TNF-receptor antagonist such as a sTNFR, a MAP-kinase inhibitor, Yohimbine, a glial cell chloride antagonist, a caspase inhibitor, an MMP inhibitor, a cannabinoid receptor agonist, arundic acid, a statin, and thalidomide or a related analog.

16. The method of claim 1, further comprising administering one or more other phosphodiesterase (PDE) inhibitors.

17. The method of claim 16, wherein the one or more PDE inhibitors is selected from the group consisting of a PDE3 inhibitor, a PDE4 inhibitor, a PDE5 inhibitor, a PDE7 inhibitor, and a PDE10 inhibitor.

18. The method of claim 1, further comprising administering one or more other agents effective for treating an affective disorder.

19. The method of claim 18, wherein the one or more agents is selected from the group consisting of an antidepressant, an antipsychotic, a tranquilizer, a sedative, a muscle relaxant, an anticonvulsant, and an insomnia therapeutic.

20. The method of claim 19, wherein the antidepressant is selected from the group consisting of a tricyclic antidepressant (TCA), a monoamine oxidase inhibitor (MAOI), a selective serotonin reuptake inhibitor (SSRI), a combined reuptake inhibitor and receptor blocker, a serotonin and norepinephrine reuptake inhibitor (SNRI), a norepinephrine and dopamine reuptake inhibitor (NDRI), and a tetracyclic antidepressant.

21. The method of claim 19, wherein the sedative is a benzodiazepine.

22. A composition comprising ibudilast, one or more other phosphodiesterase (PDE) inhibitors and a pharmaceutically acceptable excipient.

23. The composition of claim 24, wherein the one or more PDE inhibitors is selected from the group consisting of a PDE3 inhibitor, a PDE4 inhibitor, a PDE5 inhibitor, a PDE7 inhibitor, and a PDE10 inhibitor.

24. A composition comprising ibudilast, one or more other agents effective for treating an affective disorder and a pharmaceutically acceptable excipient.

25. The composition of claim 24, wherein the one or more agents is selected from the group consisting of an antidepressant, an antipsychotic, a tranquilizer, a sedative, a muscle relaxant, an anticonvulsant, and an insomnia therapeutic.

26. The composition of claim 25, wherein the antidepressant is selected from the group consisting of a tricyclic antidepressant (TCA), a monoamine oxidase inhibitor (MAOI), a selective serotonin reuptake inhibitor (SSRI), a combined reuptake inhibitor and receptor blocker, a serotonin and norepinephrine reuptake inhibitor (SNRI), a norepinephrine and dopamine reuptake inhibitor (NDRI), and a tetracyclic antidepressant.

27. The composition of claim 25, wherein the sedative is a benzodiazepine.

28. A kit comprising ibudilast, one or more other phosphodiesterase (PDE) inhibitors and instructions for use in treating an affective disorder.

29. The kit of claim 28, wherein the one or more PDE inhibitors is selected from the group consisting of a PDE3 inhibitor, a PDE4 inhibitor, a PDE5 inhibitor, a PDE7 inhibitor, and a PDE10 inhibitor.

30. A kit comprising ibudilast, one or more other agents effective for treating an affective disorder and instructions for use in treating an affective disorder.

31. The kit of claim 30, wherein the one or more agents is selected from the group consisting of an antidepressant, an antipsychotic, a tranquilizer, a sedative, a muscle relaxant, an anticonvulsant, and an insomnia therapeutic.

32. The kit of claim 31, wherein the antidepressant is selected from the group consisting of a tricyclic antidepressant (TCA), a monoamine oxidase inhibitor (MAOI), a selective serotonin reuptake inhibitor (SSRI), a combined reuptake inhibitor and receptor blocker, a serotonin and norepinephrine reuptake inhibitor (SNRI), a norepinephrine and dopamine reuptake inhibitor (NDRI), and a tetracyclic antidepressant.

33. The kit of claim 31, wherein the sedative is a benzodiazepine.
